(19) United States
(12) Patent Application Publication
(10) Pub. No.: US 2008/0208183 A1
(43) Pub. Date: Aug. 28, 2008

(54) Device for Applying a Fluid to an Area to be Treated, Comprising an Improved Discharge Nozzle

(75) Inventors: Denis Marin, Bailly (FR); Serge Sonie, La Riviere Saint-Sauveur (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Persee Medica

(21) Appl. No.: 11/919,159

(22) PCT Filed: Apr. 28, 2006
(86) PCT No.: PCT/FR2006/000967

§ 371 Date: Jan. 10, 2008

(30) Foreign Application Priority Data
Apr. 28, 2005 (FR) 05/04321
Mar. 31, 2006 (FR) 06/02832

(51) Int. Cl. A61B 18/02; B65D 83/14

(52) U.S. Cl. 606/22; 239/337; 604/310

(57) ABSTRACT

A device for applying a fluid to a localized and defined area including a reserve of fluid in the form of an aerosol; means for starting release of the fluid from the reserve; and an ejection nozzle having a conduit and an outlet orifice through which the fluid is ejected at a pressure higher than atmospheric pressure in a controlled and directed manner as regards a diameter of the application area, the fluid having a boiling point equal to or lower than −20° C., and a latent heat of evaporation equal to or lower than 200 kJ/kg.

### RELATED APPLICATION

This is a §371 of International Application No. PCT/FR2006/000967, with an international filing date of Apr. 28, 2006 (WO 2006/114532 A1, published Nov. 2, 2006), which is based on French Patent Application Nos. 05/04321, filed Apr. 28, 2005, and 06/02832, filed Mar. 31, 2006.

### TECHNICAL FIELD

This disclosure relates to dispensing a fluid contained in an aerosol and stored in a device in the form of a casing. The disclosure relates more particularly to devices that allow ejection of the fluid to be controlled at an outlet and to limit its application only to the area to be treated and to modulate its mode of action and time in a predefined fashion according to the type of application to be treated.

### BACKGROUND

U.S. Pat. No. 5,098,428 discloses a cryosurgical instrument in contact with a liquefied gas coolant contained in a thermally insulated container. When normal venting of the container is stopped, the pressure of the liquefied gas coolant can be increased in the container by a squeeze bulb. This pressure causes the liquefied gas coolant to be projected through the nozzle until normal venting is resumed. In addition, that instrument is highly sensitive to the conditions of use (atmospheric pressure, temperature, etc.) and to the remaining amount of liquefied gas, which varies over successive uses. Therefore, the characteristics of the projected gas vary from one use to the next in terms of pressure, temperature and quantity.

U.S. Pat. No. 6,296,410 discloses a device to administer a certain quantity of a liquid cooling agent. That device comprises: a container for containing the liquid cooling agent; a normally closed valve connected to the container; and operating means for temporarily opening the valve, comprising positioning means for positioning at least one part of the administering element, so that the cooling agent leaving the valve can reach the administering element. According to a first embodiment, the positioning means comprise a chamber which is connected to the valve and in which the cooling agent is inserted when the valve is operated, this chamber being designed to receive at least one part of the administering element.

Such devices do not, however, make it possible to control the ejection and application of the fluid on the area to be treated.

However, depending on the area to be treated, the jet of fluid must more or less narrow. This is even more important when the fluid is used for therapeutic, technical or cosmetic purposes. In particular, in the case of skin infections such as warts or sun spots, but also for any other application on the skin, mucous membranes, or any other part of the human or animal body, the jet required to treat the entirety of the affected area must be adapted to the surface occupied by the infection. Indeed, a jet covering an application surface that is greater than the area to be treated can treat uninfected areas, and thus causes possible damage. On the other hand, a jet partially covering the area to be treated requires repeated applications of the fluid until the infected area is completely treated. However, such repetitions contribute to making the use of such devices tiresome. They also contribute to subjecting parts of the area to be treated to multiple applications of the fluid, applications which can furthermore cause damage to the affected parts.

It could therefore be advantageous to provide a device that allows the ejection of the fluid to be controlled at the outlet of the device and to limit its application to the area to be treated and to modulate its mode of action and time in a predefined fashion according to the type of application to be treated.

### SUMMARY

We provide a device for applying a fluid to a localized and defined area including a reserve of fluid in the form of an aerosol; means for starting release of the fluid from the reserve; and an ejection nozzle having a conduit and an outlet orifice through which the fluid is ejected at a pressure higher than atmospheric pressure in a controlled and directed manner as regards a diameter of the application area, the fluid having a boiling point equal to or lower than −20° C., and a latent heat of evaporation equal to or lower than 200 kJ/kg.

### BRIEF DESCRIPTION OF THE DRAWINGS

Our device will be understood better from the following description, provided merely for the purpose of explanation, in reference to the appended drawings:

FIG. 1 shows a section view of the ejection nozzle of a fluid application device;

FIG. 2 shows a perspective view of the timer that allows the fluid to be dispensed by the device of FIG. 1, the timer being stopped;

FIG. 3 shows a top view of the timer of FIG. 2, the timer being in operation;

FIG. 4 shows a partial view of the device of FIG. 1 and the associated push button;

FIG. 5 shows a cutaway view of one side of another fluid application device;

FIGS. 6 and 7 show cutaway views of the other side of the device of FIG. 5; and

FIG. 8 shows a detail view of the system for starting the timer.

### DETAILED DESCRIPTION

We provide devices for applying a fluid to a localized and defined area, of the type comprising a reserve of fluid in the form of an aerosol and means for starting the release of the fluid from the reserve towards an ejection nozzle having a conduit and an outlet orifice through which the fluid is ejected at a pressure higher than the atmospheric pressure in a controlled and directed manner as regards the diameter aimed on the application area. The fluid has a boiling point equal to or lower than −20° C., and a latent heat of evaporation equal to or lower than 200 kJ/kg. Preferably, the fluid has a boiling point of around −24° C., and a latent heat of evaporation of around 160 kJ/kg.

The applied fluid is a cold fluid intended for therapeutic, cosmetic or technical use (cryotherapy, etc.). The device more particularly, but not exclusively, treats all kinds of skin infections such as warts, corns, growths and sun spots or any other local application on the skin, mucous membranes, tissue or any other part of a human or animal body, whether in the medical, technical or cosmetic field.

Advantageously, the fluid is kept in the reserve under conditions that allow it to be ejected at a pressure between 6 and 8 bars.

According to a preferred construction, the fluid contains difluoroethane.

The conduit has a length comprised between 3 and 9 millimeters, preferably around 6 millimeters, the outlet orifice having a diameter comprised between 0.1 and 0.8 millimeter, preferably around 0.3 millimeter. A preferred configuration, described below, includes a nozzle comprising a conduit with a length of 5.5 millimeters in which the outlet orifice has a diameter of 0.20 millimeter.

The surface of the nozzle comprising the outlet orifice has a convex, concave or flat shape.

To establish a sufficient distance between the area to be treated and the outlet of the cold fluid, but also to prevent contact between the nozzle and the application area, the device is provided, according to an advantageous configuration, with a protective cap having a sufficient height, preferably comprised between 10 and 40 millimeters. The term “height” is understood to refer to the distance comprised between the end of the conduit forming the fluid outlet and the area to be treated. A preferred configuration, as described below, will be a protective cap which, once fixed on the ejection nozzle, provides a distance with the area to be treated of 33 millimeters.

Moreover, the protective cap advantageously comprises one or more openings for venting pressure. These openings provide a sufficiently large release for the fluid in gaseous state to prevent formation of whirls inside the protective cap which would result in diverting the trajectory of the fluid when it is ejected from the nozzle. Advantageously, the opening or openings are located at the bottom of the protective cap, and preferably in the bottom quarter of the cap.

Advantageously, the protective cap has the technical characteristics required for the user to be sure of the correct operation of the control and the efficiency of the device. In particular, it is transparent, or not, to allow visual control of the action to be performed during the action to check the application and the efficiency on the area to be treated.

Advantageously, the protective cap has a tapered shape open towards the application area, the height and the solid angle of the cone being such that the base of the cone has a diameter comprised between 1 and 3 centimeters.

Advantageously, the solid angle of the protective cap is greater than or equal to that of the jet of fluid coming out from the nozzle. A solid angle of 9 degrees may be provided.

Advantageously, the localized area on which the fluid is applied matches the base of the cone.

The cap may comprise a membrane seal covering the area delimited by the cone, the membrane seal being equipped with an orifice defining the localized area for application of the fluid. Thus, only the part to be treated receives the cold fluid, the adjoining healthy parts not being damaged by the cold applied.

Moreover, to avoid any contact between the nozzle and the infected area to be treated and thus to avoid possible contagion of pathologies, the surface of the membrane seal comprising the orifice is partially raised in relation to the localized area of application. This area can be raised by around 4 millimeters.

Advantageously, the membrane seal has the technical characteristics required for the user to be sure of the correct operation of the control and the efficiency of the device. In particular, it is transparent or not, but allows visual control of the area to be treated during the application to check the efficiency of application.

The device may also comprise a timer controlling duration of releasing the fluid.

According to a particular use of the device, the area on which the fluid is applied is a localized area of skin. It can be, for example, an area of the epidermis having a skin infection, such as a wart or a sun spot, or any other cosmetic or technical requirement on a human or animal body.

In the case of the localized area covering a wart, the nozzle will advantageously have a conduit with a length of 5.5 millimeters and an outlet orifice with a diameter of 0.20 millimeter.

Likewise, it is preferable to keep the fluid in the reserve under conditions that allow it to be ejected at a pressure comprised between 6.4 and 7.6 bars. The fluid used may be in a gaseous state and is difluoroethane.

Furthermore, the surface of the nozzle, with a convex shape, advantageously has a radius of curvature of 1 centimeter.

Likewise, the protective cap, with a tapered shape, has a height of 3.3 centimeters and a solid angle of 9 degrees, and the orifice of the membrane seal has a diameter of 5 to 9 millimeters, preferably around 6 millimeters.

In the case of the area covering a sun spot, the nozzle has a conduit with a length of 3 to 9 millimeters, preferably around 6 millimeters, in which the outlet orifice has a diameter of 0.1 to 0.8 millimeters, preferably around 0.3 millimeters.

Advantageously, the surface of the nozzle is concave, with a radius of curvature of 0.2 centimeter.

Likewise, the protective cap, with a tapered shape, has a height of 2 centimeters and a solid angle of 0.12 steradians.

The following description relates to a device for dispensing a fluid allowing the application of a given amount of fluid on a localized and defined area of the skin with a defined application time with a view to treating skin infections.

The fluid used, under pressure at room temperature, is used in its liquid phase.

The fluid has a boiling point equal to or lower than −20° C., and a latent heat of evaporation equal to or lower than 200 kJ/kg. The particular combination of these two characteristics of the fluid provides optimum efficiency for the considered cryotreatments. Indeed, the boiling point makes it possible to achieve the necessary cold, and the relatively low latent heat of evaporation provides quick evaporation resulting in good transmission of cold with no risk of the fluid flowing out of the application area. Therefore, the use of the fluid makes it possible to obtain intense “dry” cold, while reducing the aggressiveness of the treatment on the application area. In one example, the fluid is of the HFC-152A type, with a boiling point of −24° C. and a latent heat of evaporation equal to 160 kJ/kg.

The quantity and duration of application of the fluid are defined according to the use for which the device is intended. When treating a wart, since intense cold is required, the area is localized in a diameter of 3 to 10 millimeters, preferably around 6 millimeters and the fluid is advantageously ejected during at least one second on the area. When treating a sun spot, since less intense cold is required, the area is localized in a diameter of 0.5 to 2 centimeters, preferably around 1.5 centimeters and the fluid is advantageously ejected during at least one second on the area.

As regards application of the fluid only on the infected area, this is obtained by the construction of the device, and more particularly by the shape and configuration of the ejection part (ejection nozzle) of the device.

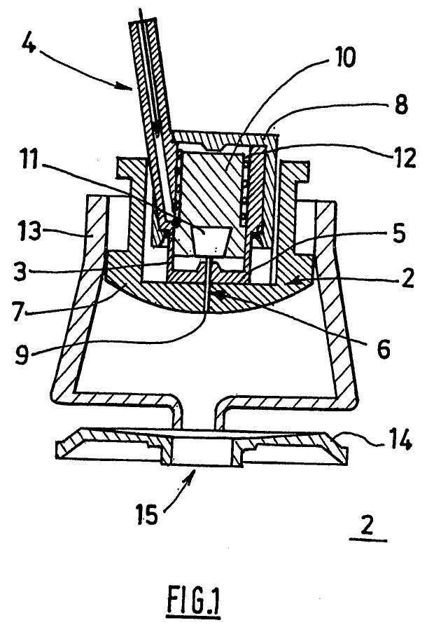

FIG. 1 shows a sectional view of such an ejection nozzle (2).

The nozzle (2) includes a hollow body in which is housed a casing (3) defining a cavity (5) intended for receiving the fluid coming from a reserve (not shown) by a conduit (4). The top part of the casing (3) is advantageously formed by a lid (8).

The nozzle (2) comprises a conduit (6) passing through the body of the nozzle (2) and the casing (3) housed in the nozzle (2).

The conduit (6) is advantageously equipped with a tubular ferrule (9), one end of the ferrule (9) opening by several millimeters into the cavity (5), the other end opening onto the ejection head (7) of the nozzle (2). The ferrule (9) has an inner diameter of 0.1 millimeters to 0.8 millimeters, preferably around 0.3 millimeters, and a length of 3 to 9 millimeters, preferably around 6 millimeters. Due to its dimensions, the ferrule (9) guarantees the flow of the jet and contributes, with the shape of the ejection head (7) of the nozzle (2), to the shape of the jet of fluid. Thus, when the area of the ejection head (7) located around and close to the outlet orifice of the ferrule (9) has a convex shape, the jet of fluid is fine, and when it has a concave shape, the jet of fluid is wide. It is therefore possible, by adjusting the shape of the ejection head (7) of the nozzle (2) and the inner diameter of the ferrule (9), to adapt the shape of the jet to the infection to be treated. It should be noted that the term “jet” is used to refer to a mix of gas/liquid.

A slide valve (10) is slidingly mounted in the cavity (5) formed by the casing (3). More specifically, the slide valve (10) is configured to slide from a position closing the end of the ferrule (9) (or conduit 6) opening into the cavity (5) (closing position) to a position leasing the end of the ferrule (9) opened to the fluid, and vice-versa. The ferrule (9) is closed by an elastomer plug (11) arranged on the bottom of the slide valve (10) in contact with the ferrule (9) to guarantee the seal.

Sliding of the slide valve (10) takes place under the action of a magnetic field controlled by a timer device described below, the slide valve (10) being made at least partially from a magnetic material. The slide valve (10) is placed in the closing position by means of a spring (12) partially arranged around the slide valve (10).

Thus, when the device (1) is in its non-operational state, the elastomer plug (11) of the slide valve (10) is pressed against the end of the ferrule (9) opening into the cavity (5) by the spring (12). The fluid coming from the reserve is then kept in the space formed between the casing (3) and the slide valve (10).

Under the action of the magnetic field arranged above the lip (8), the slide valve (10) moves from its closing position to its open position to allow evacuation via the ferrule (9) of the fluid kept trapped between the casing (3) and the slide valve (10). Movement of the slide valve (10) is stopped by the lid (8).

When the magnet is removed or placed at a sufficient distance from the lid (8), the spring (12) pushes the slide valve (10) back in the direction of the ferrule (9) until it closes the orifice of the ferrule (9) again with the elastomer plug (11) of the slide valve (12).

The device (1) is also equipped with a protective cap (13).

The protective cap (13) has the purpose of creating a sufficient distance between the area (skin) and the outlet of the cold fluid.

The protective cap (13) also prevents any contact between the ejection head (7) of the nozzle (2) and the infected area to be treated. In fact, it is imperative to avoid contagion of the parts to be treated by means of contact that might risk renewing the pathologies that are supposed to have been treated or developing other pathologies by indirect effect. For this purpose, the protective cap (13) advantageously has a height comprised between 10 and 20 millimeters.

Furthermore, to vent the gas pressure caused by ejecting the fluid, the wall of the protective cap (13) is equipped with one or more openings. The openings are preferably located in the bottom quarter of the length of the cap (13), or at least in the last third of the length of the cap (13). The number of the openings may be chosen so that the speed of the fluid at the openings is negligible compared with the ejection speed of the cold fluid.

The protective cap (13) is advantageously transparent to facilitate its positioning around the area to be treated.

Furthermore, to respect the shape of the jet at the outlet of the ejection nozzle (2), the protective cap (13) has a tapered shape open towards the area to be treated.

The device (1) may also comprise a membrane seal (14) fixed on the free end of the protective cap (13).

The membrane seal (14) limits the area to which the fluid is applied. For this reason, the membrane seal (14) comprises an orifice (15) which, when the membrane seal (14) is fixed on the end of the protective cap (13), is placed in the axis of ejection of the fluid. For wart treatment, the orifice (15) preferably has a diameter of 3 to 10 millimeters, preferably around 6 millimeters. For sun-spot treatment, it advantageously has a diameter of 5 to 20 millimeters, preferably around 15 millimeters.

The membrane seal (14) is advantageously transparent to make it easier to position the orifice (15) over the area to be treated.

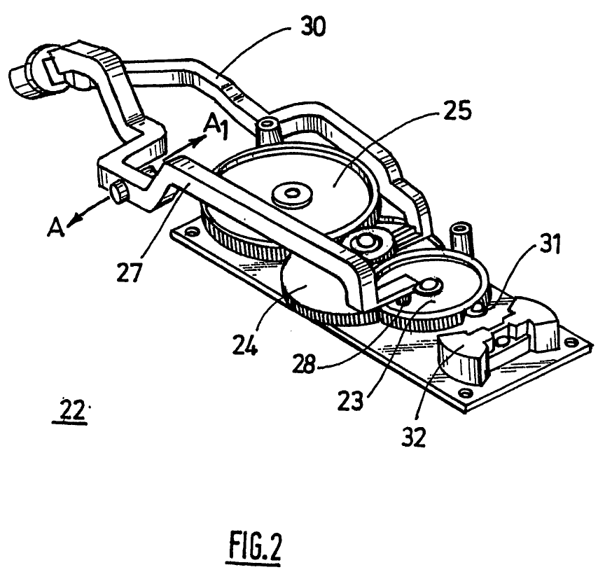

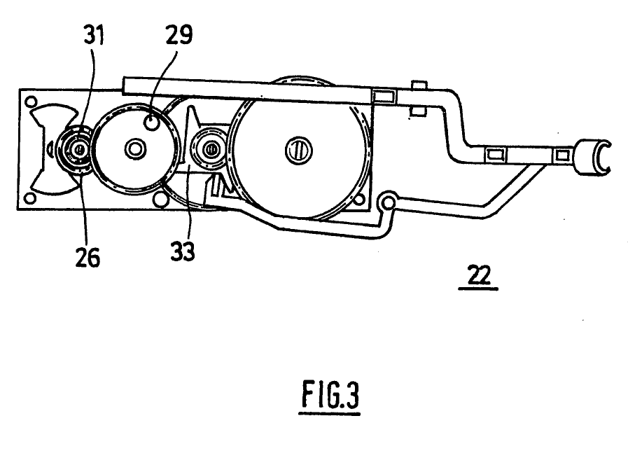

FIGS. 2 and 3 show a perspective view of the timer (22) starting the action of the magnet on the slide valve (10) of the nozzle (2), the timer (22) being respectively stopped and operational.

The term “timer” is used to refer to the elements that make up the timer itself, as well as the means implemented to trigger its operation and/or to cause it to stop.

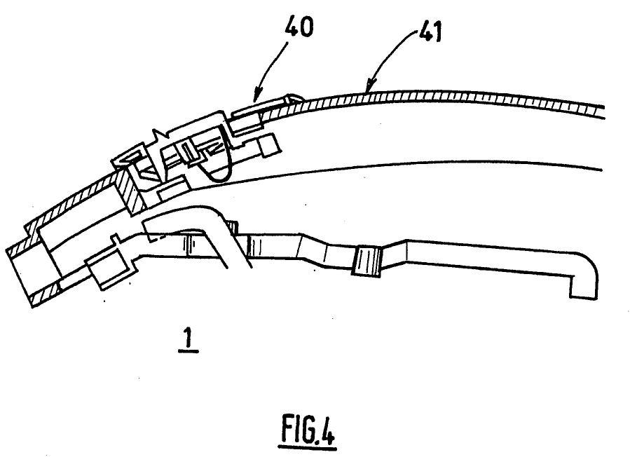

The timer (22) controls the application time of the fluid on the area to be treated by controlling the time during which the fluid is released. The fluid is released under the action of a push button (40) shown in FIG. 4, the push button (40) being slidingly mounted on the body (41) of the device (1).

The timer (22), which is mechanical, comprises gears made up of toothed wheels, including:
a first wheel (23) starting and stopping the timer (22). This wheel (23) will hereinafter be referred to as “cycle wheel.”
a second wheel (24), hereinafter referred to as “cam wheel,” starting or stopping the release of the fluid through the ejection nozzle (2).

The cycle wheel (23) is connected to the push button (40) by an arm forming a lever (27) rotatingly mounted around an axis of rotation AA1 to move from a lowered position on the cycle wheel (23) to a raised position, and vice-versa.

The arm forming a lever (27) advantageously comprises stopping means designed to keep the cycle wheel (23) stopped. The stopping means may include a lug (28) placed at the end of the arm (27), lug (28) which is configured to be housed in a hole (29) formed in the cycle wheel (23).

Thus, when the arm forming a lever (27) is in a lowered position and the lug (28) is inserted in the hole (29) of the cycle wheel (3), the cycle wheel (23) is kept stopped.

The cam wheel (24) starts or stops the release of the fluid by a release arm (30). For this purpose, the release arm (30) has one end solidly attached to the cam wheel (24), the other end being equipped with a magnetic field activating the opening or closing of the ejection nozzle (2) of the fluid reserve.

The timer (22) of the device (1) works as follows.

The arm forming a lever (27), when held in lowered position, blocks the cycle wheel (23) by the lug (28) of the arm forming a lever (27), the lug (28) being housed in the hole (29) of the cycle wheel (23). The timer (22) is then in stopped state, all the gears being kept in a blocked position.

Pressing the push button (40) triggers the rotation of the arm forming a lever (27), which passes from its lowered position on the cycle wheel (23) to a raised position, so that the lug (28) of the arm forming a lever (27) moves out of the hole (29).

Since it is no longer retained by the arm forming a lever (27), the cycle wheel (23) is then driven by a drive wheel (25) by an extension spring (not shown). For this purpose, the spring, fixed by one of its ends to the frame, is mounted coiled around the drive wheel (25). The spring is advantageously wound to have enough energy to dispense at least ten doses of the fluid, or ten treatments, a complete turn of the cycle wheel (23) corresponding to the dispensing of one dose of fluid.

Likewise, the drive wheel (25), placed in movement under the action of the spring, drives the other wheels forming the timer (22) in series.

Advantageously, the cycle wheel (23) is in contact, by an escape wheel (26) and a pinion (31) mounted on the axis of the escape wheel (26), with a metal part (32) capable of oscillating in response to a load from the cycle wheel (23). The frequency of oscillation of the metal part (32) is linked with the shape and the mass of the actual metal part, but also with the shape of the escape wheel (shape and number of teeth). The metal part (32) acts on the speed of the timer (22).

In parallel, driven by the drive wheel (25), the cam wheel (24) activates, by the cam (33) located on the cam wheel, the release arm (30) of the fluid to open and then close the nozzle (2) of the device (1).

The stroke of the timer (2) therefore ends either by automatically repositioning the lug (8) of the arm forming a lever (7) in the hole (9) of the cycle wheel (3) (when the user presses and then releases the push button (40) before the end of a treatment cycle), or by the contact of a lug formed on the wheel against the arm forming a lever (7) (in the case of the user continuously pressing the push button).

Indeed, in the case of the user continuously pressing the push button (40), thus keeping the end of the arm forming a lever (27) in a raised position, and therefore keeping the timer (22) in a permanent state of operation until the push button is released (40), the timer (22) advantageously comprises secondary safety stopping means (not shown). The safety stopping means are supported by the cycle wheel (23). More precisely, the safety stopping means include a lug formed on the surface of the cycle wheel (23) intended to come into contact with the arm forming a lever (27). The lug is arranged on the cycle wheel (23) and is sized so that, when the arm forming a lever (27) is held in a raised position, the lug comes to a stop against the latter.

Thus, regardless of the use given to the device, the latter is such that a single dose can only be applied to the area once the push button (40) has been pressed for the first time (continuously or not), the dose being applied until the end of dispensing the quantity and the pre-determined time of the fluid.

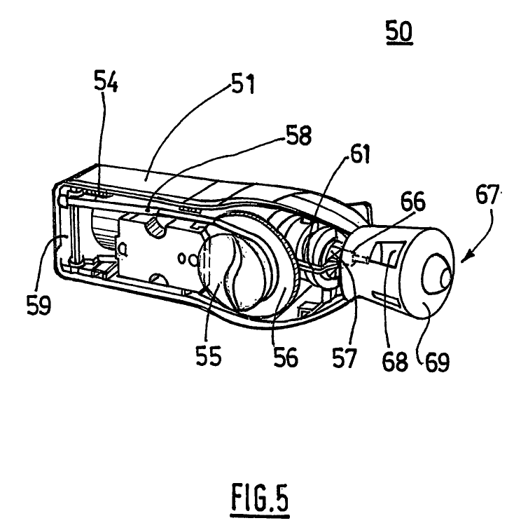

In reference to FIGS. 5 to 8, another device for applying a fluid (50) is described.

The device (50) is formed by two shell halves (51, 52) snap fitted onto one another, each of these shell halves forming a lateral side of the device (50).

In the example shown, the snap fitting is performed by six tongues (53) distributed along the edge of the shell half (52), on the inner surface, tongues (53) which are respectively received in a housing (54) made and positioned for this purpose on the inner surface of the shell (51). The housings (54) and the tongues (53) are configured not to allow, or to greatly complicate, the removal of the tongues once the two shell halves are snap fit together.

A container (65) forming the fluid reserve is housed between these two shell halves (51, 52).

The container (65) comprises a bag and a crimped pump which, when pressed, releases the gas (not shown). The gas chosen, in this case R152A, is injected into the bag and between the walls of the bag and of the container, to create, with the air surrounding the bag, pressure between the walls that is higher than the balance pressure (or saturation vapor pressure) of the R152A in the bag.

The ratio of these pressures may be determined according to the desired speed of ejection of the gas. Care will be taken, however, to avoid too great a difference between them to prevent too quick a diffusion of the gas. Thus, preferably, a mass of 2.5 g (corresponding to a pressure comprised between 6.2 bars and 7.6 bars at 20° C.) may be provided between the walls of the bag and of the container, and of 9 g (corresponding to a pressure comprised between 8.0 and 9.5 bars at 20° C.) inside the bag, to allow the delivery of 13 ejections of gas, and therefore of 13 doses of treatment, with the pressure in the bag ranging from 7.6 bars for the first ejection to 6.4 bars for the 13^{th} ejection.

The container (65) is held between the shell halves (51, 52), in a reception casing (60) provided for such purpose.

The casing (60) is rotatingly mounted on the edge of the half wall at the bottom of the shell (52) around a transverse axis AA1.

The nozzle of the device (50) is formed by a spray (66), the outlet orifice of which advantageously has a diameter of 0.20 millimeters.

The dimensions and shape of the spray (66) are chosen to allow control over the rate of flow and the direction of the gas. More specifically, the dimensions of the spray (66) are defined according to the gas used and its properties, as well as the pressure maintained in the bag of the container (65). The aim is to obtain an ejection of gas in a very short time, around three seconds, cooling the skin to around −5°, in such a way as to produce a thermal shock in several seconds (around five seconds) without pain.

The device (50) also comprises, like the previously described device, a transparent protective cap (67), the wall of which is provided with openings (67) for venting the pressure of the gas, and the end of which is provided with a membrane seal (69).

These openings (68) are advantageously located in the bottom half of the cap (67), which is to say the half closest to the skin when the cap is placed against the skin. The openings (68) are sized and arranged to allow venting of enough gas to prevent formation of whirls inside the cap (67) which could divert the trajectory of the jet.

Advantageously, the length of the protective cap (67) depends on the optimum distance between the outlet of the spray (66) and the skin, or 33 millimeters: any shorter and the speed of the fluid causes the fluid to be projected onto the walls of the protective cap, thus generating a loss of fluid; any longer and the evaporation is excessive.

FIG. 5 shows the device (50) in which the shell half (52) has been removed to show a part of the system for starting the timer.

The system for starting the timer comprises a resetting handle (55) solidly attached to a wheel (56), hereinafter called “cam wheel.” Like the cam wheel (24) in the previously described structure, the cam wheel (56) has the aim of starting or stopping the release of the fluid. This activation is described below.

A spring (58), designed to supply the energy required to dispense a dose of the fluid, is fixed, coiled, by one of its ends to the cam wheel (56), the other end being fixed to the surface (59) of the shell half (51) forming the half wall of the bottom of the device (50). It is held between the resetting handle (55) and the cam wheel (56).

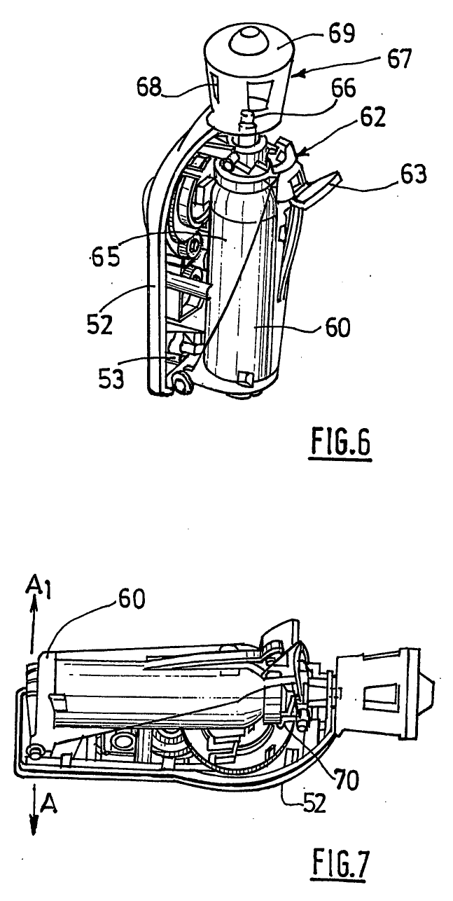

FIGS. 6 and 7 show the device (50) in which the shell half (51) has been removed to show the means implemented to activate the cam wheel (56), and thus to start or stop the release of the fluid.

The means comprise a starting lever and a rocking lever (61).

The starting lever is formed by the casing (60), the rotating movement of which is activated by a starting button (63). The starting lever is hereinafter referred to with the number “60.”

The rocking lever (61) is arranged so that one of its ends (70) remains in contact with the cam wheel (56). The latter therefore has one surface on which the resetting handle (55) is mounted, the other surface being configured to accommodate the friction end (70) of the rocking lever (61).

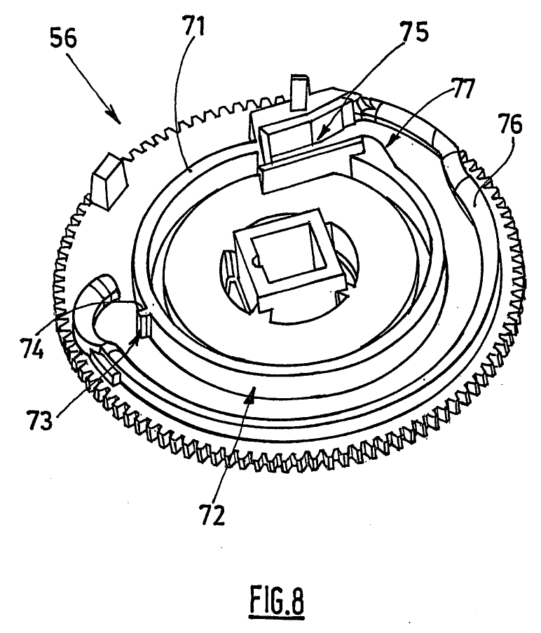

FIG. 8 shows a front view of the cam wheel (56) designed to accommodate the friction end (70) of the rocking lever (61). For this purpose, the cam wheel (56) comprises a guiding rib (71) intended to guide the rocking lever (61) on its outer periphery (72). The outer periphery (72) of the guiding rib (71) is equipped with an extension (73) forming a boss in front of the locking tab for the rocking lever (61) when resetting the handle (55). The rocking lever (61) is blocked by the face (74) of the extension (73).

The cam wheel (56) also comprises a stopping tab (75) intended to stop the rocking lever (61) in its movement, the stopping tab (75) being preceded by a boss (77). The movement imposed on the rocking lever (61) by the boss (77) stops the dispensing of the fluid by means of the hook-shaped part (63). The stopping of the rocking lever (61) by the stopping tab (75) causes the starting lever (60) to lock.

Advantageously, the stopping tab (75) is U-shaped.

The cam wheel (56) may comprise a second rib (76) which maintains and guides the rocking lever (61) between the two ribs (71, 76). This second rib (76) has the advantage of not requiring the spring effect required for a rocking lever (61) when the cam wheel (56) only comprises one rib (71).

The rocking lever (61) is solidly attached to the starting lever (60) by means of a hook-shaped part (62).

The timer associated with the cam wheel (56) comprises the following six parts: one plate, one pallet, two unbalanced masses and two pinions.

In addition, it is evident that the resetting handle (55) and the starting button (63) are arranged to extend to the outside of the shell halves (51, 52).

The operation of the fluid dispensing system is as follows.

The resetting handle (55) is turned to start the time, driving the cam wheel (56) through its movement. The handle (55) is turned until the rocking lever (61) becomes locked against the extension (73) that constitutes the blocking tab.

At the same time, the spring (58), held between the resetting handle and the cam wheel, is driven by the movement of the cam wheel (56), coiling onto itself.

Preferably, this activation is performed by turning the resetting handle (55) through half a turn.

The fluid application device (50) is then ready to be used to dispense a defined dose of fluid over a given period of time.

The fluid dispensing operation is launched by manually pressing the starting button (63) towards the top of the device (50) in the direction of the nozzle. Through its movement, the starting button (63) drives the starting lever formed by the casing (60) in a rotating movement in the direction of the shell (52). Through its movement, the starting lever (60) drives the lowering of the hook-shaped part (63), which lifts the rocking lever (61) to allow the passage of the extension (72). Since the rocking lever (61) is no longer blocked with the cam wheel (56), the latter is placed in rotation under the action of the spring (58). The timer is then started.

At the same time, the starting lever (60) rotates and comes to a rest against the bottom of the bag, which results in opening the crimped pump in the container (65), which releases the gas.

The gas is then dispensed until the rocking lever (61) is placed in contact with the boss (77) provided on the cam wheel (56). The cam wheel (56) continues its movement of rotation until the rocking lever (61) reaches the stopping tab (75). The movement of the starting lever (60) is then locked.

To operate a new dispensing fluid, a further step of resetting the timer needs to be performed, the arm of the rocking lever (61) going from its stopped position to its intermediate locking position under the action in the opposite direction of the tension exerted by the spring (58), and so on.

Our devices are described above as examples. It is understood that those skilled in the art are capable of creating different variations without departing from the scope of the appended claims.

1-34. (canceled)

35. A device for applying a fluid to a localized and defined area comprising:
a reserve of fluid in the form of an aerosol;
means for starting release of the fluid from the reserve; and
an ejection nozzle having a conduit and an outlet orifice through which the fluid is ejected at a pressure higher than atmospheric pressure in a controlled and directed manner as regards a diameter of the application area, the fluid having a boiling point equal to or lower than −20° C., and a latent heat of evaporation equal to or lower than 200 kJ/kg.

36. The device according to claim 35, wherein the fluid has a boiling point of around −24° C., and a latent heat of evaporation of around 160 kJ/kg.

37. The device according to claim 35, wherein the fluid is kept in the reserve to be ejected at a pressure between 6 and 8 bars.

38. The device according to claim 35, wherein the fluid comprises difluoroethane.

39. The device according to claim 35, wherein the conduit has a length between 3 and 9 millimeters and the outlet orifice has a diameter between 0.1 and 0.8 millimeters.

40. The device according to claim 35, wherein the conduit has a length of 5.5 millimeters and the outlet orifice has a diameter of 0.20 millimeters.

41. The device according to claim 35, wherein a surface of the nozzle comprising the outlet orifice has a convex or concave shape.

42. The device according to claim 35, further comprising a protective cap having a height sufficient to prevent contact between the nozzle and the application area.

43. The device according to claim 42, wherein the height of the protective cap is between 10 and 40 millimeters.

44. The device according to claim 42, wherein the height of the protective cap is 33 millimeters.

45. The device according to claim 42, wherein the protective cap comprises at least one opening for venting pressure.

46. The device according to claim 45, wherein the opening or openings are located at a bottom portion of the protective cap.

47. The device according to claim 42, wherein the protective cap has a tapered shape open towards the application area, and the height and a solid angle of the tapered shape are such that the base of the cone has a diameter between 1 and 3 centimeters.

48. The device according to claim 47, wherein the solid angle of the protective cap is greater than or equal to that of the jet of fluid coming out from the nozzle.

49. The device according to claim 47, wherein the protective cap has a solid angle of 9 degrees.

50. The device according to claim 47, wherein the localized area on which the fluid is applied matches the base of the tapered shape.

51. The device according to claim 42, wherein the protective cap comprises a membrane seal covering an area delimited by the tapered shape, the membrane seal being equipped with an orifice defining a localized area for application of the fluid.

52. The device according to claim 51, wherein the surface of the membrane seal comprising the orifice is partially raised in relation to the localized area of application.

53. The device according to claim 52, wherein the area is raised by around 4 millimeters.

54. The device according to claim 35, wherein the area is located on the skin, mucous membranes, tissue or any other part of a human or animal body, for medical, technical or cosmetic applications.

55. The device according to claim 54, wherein the area covers a wart.

56. The device according to claim 55, wherein the nozzle has a conduit with a length of 5.5 millimeters and the outlet orifice has a diameter of 0.20 millimeter.

57. The device according to claim 55, wherein the fluid is kept in the reserve to be ejected at a pressure between 6.4 and 7.6 bars.

58. The device according to claim 55, wherein the fluid comprises difluoroethane.

59. The device according to claim 55, wherein a surface of the nozzle is convex, with a radius of curvature of 1 centimeter.

60. The device according to claim 55, wherein the protective cap, with a tapered shape, has a height of 3.3 centimeters and a solid angle of 9 degrees.

61. The device according to claim 55, wherein the orifice of the membrane seal has a diameter of around 5 to 9 millimeters.

62. The device according to claim 54, wherein the area covers a sun spot.

63. The device according to claim 62, wherein the nozzle has a conduit with a length of 3 to 9 millimeters in which the outlet orifice has a diameter of 0.1 to 0.8 millimeters.

64. The device according to claim 62, wherein a surface of the nozzle is convex, with a radius of curvature of around 15 millimeters.

65. The device according to claim 62, wherein the protective cap, with a tapered shape, has a height between 2 and 3 centimeters and a solid angle of 0.12 stearadians.

66. The device according to claim 35, further comprising a timer controlling the time during which the fluid is released.
